(19) United States
(12) Patent Application Publication
(10) Pub. No.: US 2017/0049342 A1
(43) Pub. Date: Feb. 23, 2017

(54) BLOOD PRESSURE MONITOR

(71) Applicant: SYNCMOLD ENTERPRISE CORP., New Taipei City (TW)

(72) Inventors: Yao-Lin CHEN, New Taipei City (TW); Chung-Cheng HU, New Taipei City (TW); Cheng-Chieh HUNG, New Taipei City (TW)

(21) Appl. No.: 15/136,061

(22) Filed: Apr. 22, 2016

(30) Foreign Application Priority Data
Aug. 19, 2015 (TW) 104213321

(51) Int. Cl. A61B 5/022; A61B 5/0225

(52) U.S. Cl. CPC A61B 5/02233; A61B 5/0225

(57) ABSTRACT

A blood pressure monitor comprising a main engine and a cuff is provided. The main engine has a housing, a pump which is disposed in the housing, and a plurality of first venting pipes. A plurality of first through-holes is formed on the housing to gas-communicate with the pump. The cuff has a gasbag and a plurality of gas nozzles. The gas nozzles extend from the gasbag to connect with the first venting pipes. Gas generated from the pump inflates the gasbag through the first through-holes, the first venting pipes, and the gas nozzles.

### CROSS REFERENCE TO RELATED APPLICATION

This application claims the benefits of the Taiwan Patent Application Serial Number 104213321, filed on Aug. 19, 2015, the subject matter of which is incorporated herein by reference.

### BACKGROUND OF THE INVENTION

1. Field of the Invention

The present invention relates to a blood pressure monitor with an improved structure, the main engine and the cuff of which may be fastened tightly to each other.

2. Description of Related Art

FIG. 1 illustrates an integrated blood pressure monitor 1 known in the art, which comprises a main engine 11 and a cuff 12, wherein the main engine 11 and the cuff 12 are fixed to each other inseparably. The cuff 12 has a plurality of convex soft connecting tubes 121, and the main engine 11 has a plurality of corresponding through-holes, wherein each of the connecting tubes 121 passes through the corresponding through-hole 112 respectively and extends to the inside of the main engine 11 for connecting a pump 111. The pump 111 may inflate the cuff 12 through the connecting tubes for measuring blood pressures.

During the assembling process of the aforementioned blood pressure monitor 1 known in the art, each of the connecting tubes 121 should pass through the through-holes 112 and then connect to the pump 111. However, since the cuff 12 is made of soft materials, and the housing (unnumbered in figure) of the main engine 11 is blocked in the middle so that the precise position of the pump 111 cannot be located from the outside of the housing of the main engine 11. Therefore, it is difficult to locate the connecting tubes 121 and the pump 111 for their connection; also, it is difficult to make sure that the connection thereof is tightly connected.

Accordingly, the present invention provides a novel blood pressure monitor with a main engine and a cuff that are tightly fixed to each other, during the assembling process, in order to improve the structural quality of the blood pressure monitor which is desirable and has potential in the technical field.

### SUMMARY OF THE INVENTION

One object of the present invention is to provide a blood pressure monitor, wherein the pump in the main engine may be precisely connected to the gasbag of the cuff in order to efficiently inflate the gasbag of the cuff, and the gasbag of the cuff may be tightly fixed to the main engine during inflation.

For achieving the aforementioned object, the present invention provides a blood pressure monitor, which comprises a main engine and a cuff. The main engine includes a housing; a pump; a relief valve; and a manifold, wherein the housing has two sets of a first through-hole, a first venting pipe extending outwardly, and a second venting pipe extending inwardly, wherein the first through-hole, the first venting pipe, and the second venting pipe in each set are interconnected to each other. The manifold is sleeved to the second venting pipes and being interconnected to the pump and the relief valve. The cuff includes a cloth cover, a gasbag, and two gas nozzles, wherein the gasbag is covered by the cloth cover and has a capsule. The gas nozzles extend from the capsule and pass through the cloth cover and are connected to the first venting pipes respectively.

When the pump is pumping and the relief valve is closed, the capsule will become a closed chamber and the air generated from the pump will flow sequentially through the manifold, the second venting pipe, the first through hole, the first venting pipe, and the gas nozzle. When the pump stops pumping and the relief valve is opened, the air in the capsule will deflate from the relief valve through the gas nozzle, the first venting pipe, the first through-hole, the second venting pipe, and the manifold.

According to one preferred embodiment of the present invention, the first venting pipes and the second venting pipes are hollow tubes made of rigid materials. Two notches are formed separately on the housing corresponding to the first venting pipes. The gas nozzles respectively engage to the notches when the gas nozzles connect to the first venting pipes. Each of the gas nozzles has a stopper, which abuts against an outer surface of the cloth cover when the gas nozzles pass through the cloth cover. The cuff further comprises a surrounding body, which is disposed adjacent to the gasbag and covered by the cloth cover, and the gas nozzles pass through the surrounding body. The cuff further comprises a board body, which is formed with two second through-holes, and the gas nozzles are integrally formed on the board body and connected to the second through-holes respectively.

According to another preferred embodiment of the present invention, the blood pressure monitor further comprises a first fixed element, wherein the housing has a first fixing hole, and the cuff further comprises a second fixing element having a second fixing hole. The second fixing element pierces through the capsule, the surrounding body, and the cloth cover from the board body, wherein the first fixing element is fixed to the first fixing hole and the second fixing hole from the inside of the housing. In addition, the cuff has at least one hook, the housing is formed with at least one concave hole which is engaged with the hook.

Furthermore, the main engine may communicate wirelessly with an electronic device when operating the blood pressure monitor.

Other objects, novel features, and advantages of the invention will become more apparent from the following detailed description when taken in conjunction with the accompanying drawings.

### BRIEF DESCRIPTION OF THE DRAWINGS

FIG. 1 is a sectional view of a known blood pressure monitor;

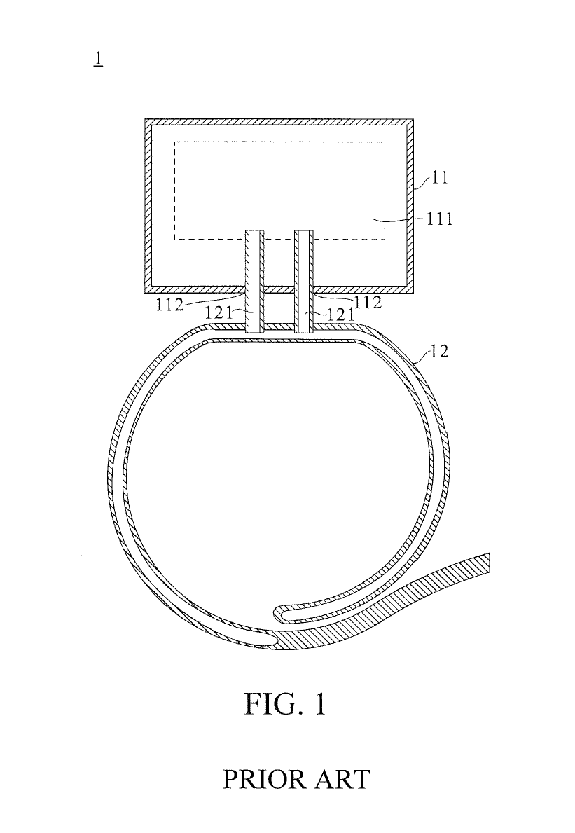

FIG. 2 is an operational perspective view of the blood pressure monitor of the present invention;

FIG. 3 is a bottom perspective view of the main engine of the blood pressure monitor of the present invention;

FIG. 4 is an internal structure perspective view of the main engine of the blood pressure monitor of the present invention;

FIG. 5 is a perspective view of the cuff of the blood pressure monitor of the present invention; and

FIG. 6 is a partial section view of the blood pressure monitor of the present invention.

### DETAILED DESCRIPTION OF THE PREFERRED EMBODIMENT

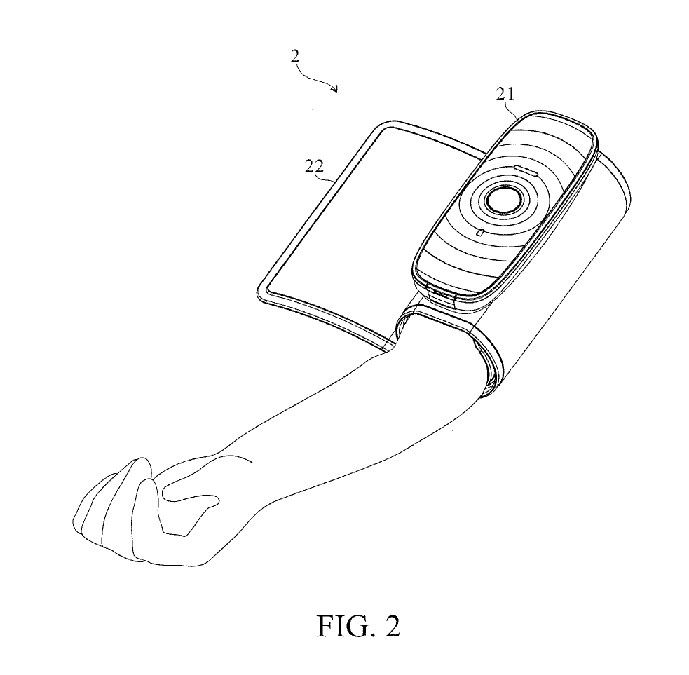

Please refer to FIG. 2, the blood pressure monitor 2 of the present invention comprises a main engine 21 and a cuff 22, wherein the main engine 21 and the cuff 22 are fixed to each other. When a user is operating the blood pressure monitor 2 of the present invention, the cuff 22 is winded on his/her arm, and the main engine 21 may communicate wirelessly with an electronic device (not shown in figures) such as a mobile phone, and the blood pressure monitor 2 of the present invention may be actuated by APPs for the blood pressure measurement. The result of the blood pressure measurement may be sent from the main engine 21 and display(s) on the electronic device. The communication method between the blood pressure monitor and the electronic device is not limited thereto, for example, the communication may be made using a connecting wire.

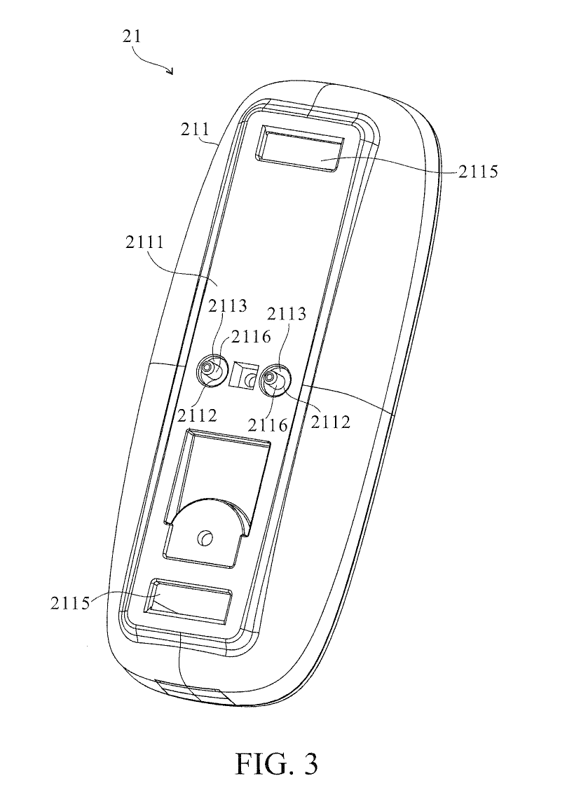

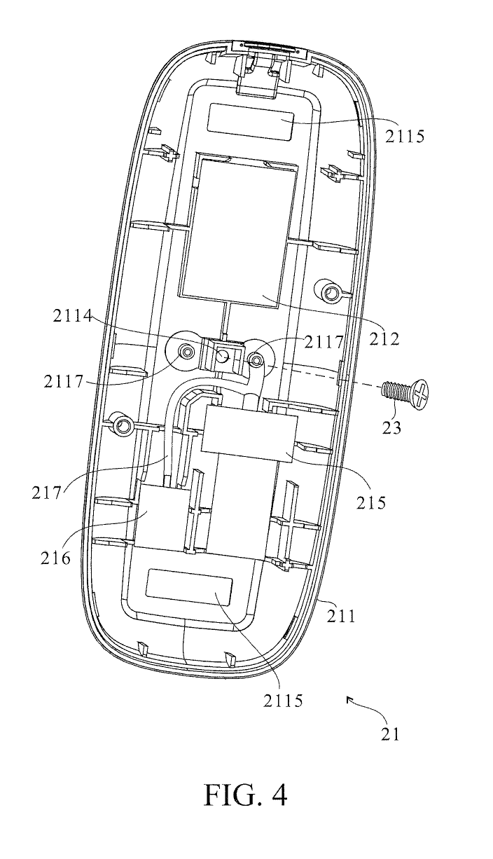

Please refer to FIG. 3 and FIG. 4, wherein FIG. 3 illustrates the outer bottom of the main engine 2 and FIG. 4 illustrates the internal structure of the main engine 21. The main engine 21 includes a housing 211, a battery 212 contained in the housing 211, a pump 215, a relief valve 216, a manifold 217, and a circuit board (not shown in figure). The housing 211 has two sets of a first through-hole 2112, a first venting pipe 2116 extending outwardly, and a second venting pipe 2117 extending inwardly, wherein the first through-hole 2112, the first venting pipe 2117, and the second venting pipe 2117 in each set are interconnected to each other. The battery 212 is electrically connected to the circuit board, and the circuit board is further electrically connected to the pump 215 and the relief valve 216 respectively. The pump 215 and the relief valve 216 are connected through the manifold 217 and the manifold 217 is sleeved to one of the second venting pipes 2117 for connecting one of the first through-hole 2112. In the present invention, another first through-hole 2112 is connected to a pressure sensor (not shown in figures) by a soft tube (not shown in figures) for obtaining the pressure value of the inflation and assuring the formation of a closed gas loop. The first venting pipes 2116 and the second venting pipes 2117 are hollow tubes made of rigid materials, such as a mixed material of ABS and PC, and are disposed correspondingly to the first through-holes 2112. Accordingly, the second venting pipes 2117 facilitate the manifold 217, made of soft material such as rubber, to be sleeved thereto. Also, the pump 215 and the relief valve 216 do not block the second venting pipes 2117 when assembling, so that the convenience of assembling the blood pressure monitor may be achieved.

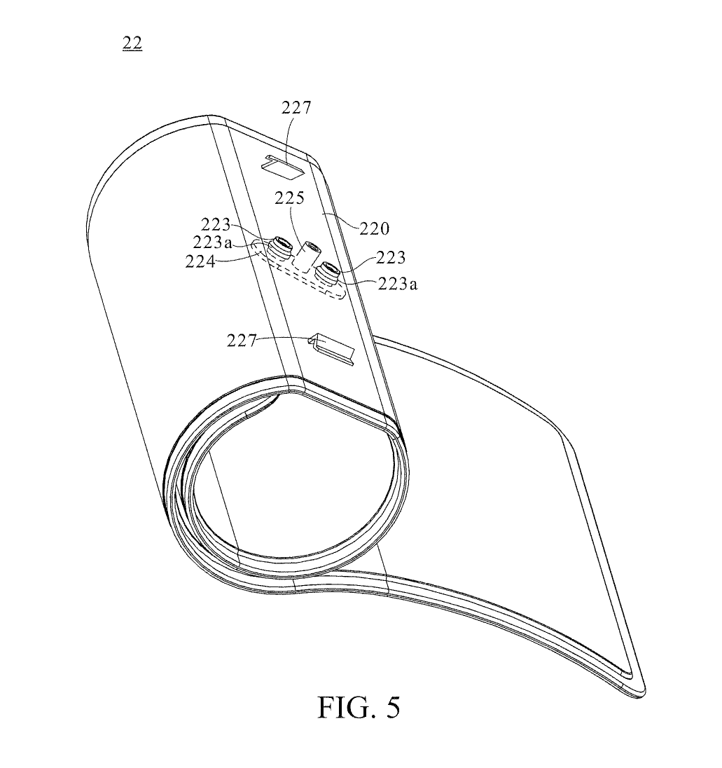

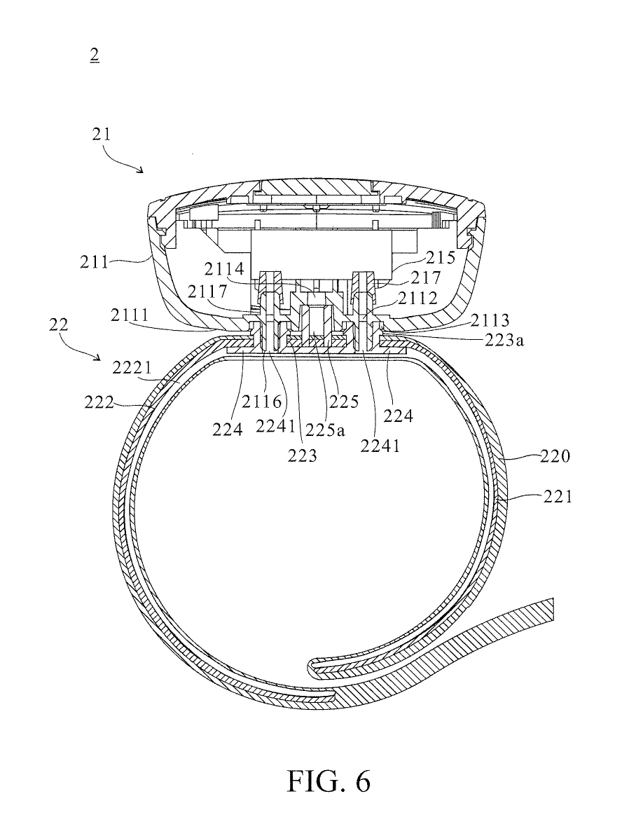

Accordingly, please refer to FIG. 5 and FIG. 6, the cuff 22 includes a cloth cover 220, a surrounding body 221, a gasbag 222, and two gas nozzles 223. In the present embodiment, the surrounding body 221 is a winding plastic plate with a C-shaped cross-section. The gasbag 222 is disposed adjacent to the surrounding body 221, both of the surrounding body 221 and the gasbag 222 are covered by the cloth cover 220. The gasbag 222 has a capsule 2221 which becomes a closed chamber when the relief valve 216 is closed. The gas nozzles 223 extend from the capsule 2221 and pass through the cloth cover 220 and the surrounding body 221 for connecting the first venting pipes 2116. Since the first venting pipes 2116 are hollow tubes made of rigid materials, the assembling procedure of the gas nozzles 223 becomes easier and more accurate. Thereby, when the blood pressure monitor 2 of the present invention is applied for blood pressure measurements, the circuit board releases signals for controlling the inflation of the pump 215 and closing the relief valve 216. After the gas generated by the pump 215 passes through the manifold 217, the second venting pipe 2117, the first through-hole 2112, the first venting pipe 2116, and the gas nozzle 22, the gas fills the capsule 2221 of the gasbag 222 to finish the inflation of the blood pressure monitor 2. After the measurement of the blood pressure is made, the circuit board releases signals for stopping the inflation of the pump 215 and opening the relief valve 216. On the contrary, the gas in the capsule 2221 passes through the gas nozzle 223, the first venting pipe 2116, the first through-hole 2112, the second venting pipe 2117 and the manifold 217, and deflates to the outside through the relief valve 216 to finish the deflation process of the blood pressure monitor 2.

Please refer to FIG. 3 and FIG. 6, two notches 2113 are formed respectively on the housing 211 and correspondingly to the first venting pipes 2116. One end of each gas nozzles 223 is tightly engaged to each of the notches 2113 for preventing gas leakage.

Please refer to FIG. 5 and FIG. 6, each gas nozzles 223 has a stopper 223a with an annulus shape. In detail, the stopper 223a abuts against the outer surface of the cloth cover 220 when the gas nozzles pierce through the surrounding body 221 and the cloth cover 220 from the capsule 2221 for assuring at least a portion of the gas nozzle 223 may be exposed from the cloth cover 220.

In the present embodiment, the cuff 22 further comprises a board body 224 and a second fixing element 225. The board body 224 is disposed in the capsule 2221 and formed with two second through-holes 2241. The gas nozzles 223 and the second fixing element 225 are integrally formed on the board body 224, and the gas nozzles 223 interconnect to the second through-holes 2241. The surrounding body 221 and the cloth cover 220 may be clamped by the board body 224 and the stopper 223a for preventing the detachment and assuring that at least a portion of the gas nozzle 223 may be exposed from the cloth cover 220 and that the gas nozzles 223 may be firmly sleeved to the first venting pipes 2116 when assembling the blood pressure monitor 2. As illustrated in FIG. 6, a portion of the gasbag 222 is clamped between the board body 224 and the surrounding body 221, wherein the junction of the board body 224 and the gasbag 222 is jointed by the high-frequency method.

Please refer to FIG. 4, FIG. 5 and FIG. 6, the blood pressure monitor 2 further comprises a first fixing element 23; the housing 211 has a first fixing hole 2114; and the second fixing element 225 has a second fixing hole 225a. Since the second fixing element 225 pierces through the capsule 2221, the surrounding body 221, and the cloth cover 220; the first fixing element 23 may extend through the housing 211 and be fastened to the first fixing hole 2114 and the second fixing hole 225a. Accordingly, the attachment of the cuff 22 to the housing 211 of the main engine 21 can be ensured, therefore, the gasbag 222 will be no concern about separating from the main engine 21 due to the deformation of the gasbag 222 when the gas inflates the capsule 2221 of the gasbag 222 and inflates the gasbag 222.

Preferably, the cuff 22 has two hooks 227, and the housing 211 is formed with two concave holes 2115, which are engaged with the hooks 227 for reinforcing the engaging strength between the cuff 22 and the main engine 21. However, the number of the hooks and the concave holes are not particularly limited in other embodiments of the present invention.

In summary, the cuff 22, the pump 215 and the relief valve 216 are fastened to the housing 211 of the blood pressure monitor 2. With the first venting pipes 2116 and the second venting pipes 2117 made of rigid materials and arranged with the manifold 217, these components will not be blocked and can be securely connected when assembling the blood pressure monitor. In addition, the cuff 22 may be firmly attached to the main engine 21 for preventing the detachment of the cuff 22 during the inflation by the first fixing element 25 which is fastened to the first fixing hole 2114 and the second fixing hole 225a. Accordingly, the quality of the blood pressure monitor 2 can be improved and the production cost can be decreased.

Although the present invention has been explained in relation to its preferred embodiment, it is to be understood that many other possible modifications and variations can be made without departing from the spirit and scope of the invention as hereinafter claimed.

What is claimed is:

1. A blood pressure monitor, comprising:
a main engine, including a housing, a pump, a relief valve, and a manifold, wherein the housing has two sets of a first through-hole, a first venting pipe extending outwardly, and a second venting pipe extending inwardly, wherein the first through-hole, the first venting pipe, and the second venting pipe in each set are interconnected to each other, the manifold is sleeved to one of the second venting pipes and being interconnected to the pump and the relief valve; and
a cuff, including:
a cloth cover,
a gasbag covered by the cloth cover and having a capsule; and
two gas nozzles extending from the capsule and passing through the cloth cover, and connected to the first venting pipes;
wherein, when the pump is pumping and the relief valve is closed, the capsule becomes a closed chamber and air generated from the pump flows sequentially through the manifold, the second venting pipe, the first through-hole, the first venting pipe, and the gas nozzle; and when the pump stops pumping and the relief valve is opened, the air in the capsule is deflated from the relief valve through the gas nozzle, the first venting pipe, the first through-hole, the second venting pipe, and the manifold.

2. The blood pressure monitor as claimed in claim 1, wherein two notches are formed separately on the housing and correspondingly to the first venting pipes, and the gas nozzles respectively engage to the notches when the gas nozzles connect to the first venting pipes.

3. The blood pressure monitor as claimed in claim 2, wherein each of the gas nozzles has a stopper, which abuts against an outer surface of the cloth cover when the gas nozzles pierce through the cloth cover.

4. The blood pressure monitor as claimed in claim 3, wherein the cuff further comprises a surrounding body, which is disposed adjacent to the gasbag and covered by the cloth cover, and the gas nozzles pierce through the surrounding body.

5. The blood pressure monitor as claimed in claim 4, wherein the cuff further comprises a board body, which is formed with two second through-holes, and the gas nozzles are integrally formed on the board body and connect to the second through holes respectively.

6. The blood pressure monitor as claimed in claim 5, further comprises a first fixing element, wherein the housing has a first fixing hole; the cuff further comprises a second fixing element having a second fixing hole; and the second fixing element piercing through the capsule, the surrounding body, and the cloth cover from the board body, wherein the first fixing element is fixed to the first fixing hole and the second fixing hole from the inside of the housing.

7. The blood pressure monitor as claimed in claim 1, wherein the cuff has at least one hook, the housing is formed with at least one concave hole which is engaged with the hook.

8. The blood pressure monitor as claimed in claim 1, wherein the main engine communicates wirelessly with an electronic device.

9. The blood pressure monitor as claimed in claim 1, wherein the first venting pipes and the second venting pipes are hollow tubes made of rigid materials.
